(19) United States
(12) Patent Application Publication
(10) Pub. No.: US 2012/0107841 A1
(43) Pub. Date: May 3, 2012

(54) Serum Diagnostic Method, Biomarker and Kit for Early Detection and Staging of Alzheimer's Disease

(76) Inventor: John A. McIntyre, Indianapolis, IN (US)

(21) Appl. No.: 13/186,017

(22) Filed: Jul. 19, 2011

Related U.S. Application Data
(60) Provisional application No. 61/365,550, filed on Jul. 19, 2010.

(51) Int. Cl. G01N 33/566; C07K 16/18

(52) U.S. Cl. 435/7.92; 530/389.1

(57) ABSTRACT

A laboratory method for screening, diagnosing, monitoring and/or staging early onset Alzheimer's disease which consists of mild cognitive impairment entails conducting a blood test after an oxidative exposure of serum to assay for the presence of an elevated level of redox-reactive autoantibodies.

### CROSS-REFERENCE TO RELATED APPLICATION

This application is a non-provisional application claiming benefit of the filing date of provisional application No. 61/365,550, filed Jul. 19, 2010, the entire contents of which are incorporated hereon by reference.

### BACKGROUND OF THE INVENTION

Alzheimer's disease (AD) is a currently incurable progressive chronic neurodegenerative disease characterized by severe cognitive decline, generally afflicting the elderly. AD is the 7^{th} leading cause of all deaths in the US and 5^{th} leading cause of death in Americans aged greater than 65. In 2009 12.5 billion hours of care was given to AD patients, at a cost of ˜$144 billion. By 2050 the numbers of people suffering from dementia is expected to approach 16 million in the US alone, absorbing hundreds of billions dollars in healthcare and related costs. By 2050 the incidence of AD is expected to approach nearly 1 million people/year with a total estimated prevalence of 11-16 million [2010 Alzheimer's disease facts and figures. Alzheimers Dement 6, 158-194 (2010).]. No treatment is available to prevent neurodegeneration. Existing symptomatic therapies temporarily slow cognitive decline. New therapies in clinical trials target various selective biochemical pathways hypothesized to be either necessary or sufficient for disease etiology. Since AD is really a ‘syndrome’ rather than a ‘disease’ multiple therapeutic modalities and approaches are likely to emerge from future clinical trial data. Drugs that promise to cure Alzheimer's disease are in clinical development but their effectiveness will rely on early diagnosis of the disease. It is presumed that therapeutic approaches that identify a novel serological biomarker for early detection of disease onset will be a key component of maximizing therapeutic efficacy, [Samgard, K., et al. Cerebrospinal fluid total tau as a marker of Alzheimer's disease intensity. Int J Geriatr Psychiatry 25:403-410 (2010)]. ‘Sporadic’ AD (99%) arises as a result of multiple factors, such as age, family history and others. Only 1% of cases are the result of established genetic variations. At present there is no universally accepted serum biomarker of early sporadic AD disease progression. The sensitivity of CSF biomarker measurements [Hu, W. T., et al. Novel CSF biomarkers for Alzheimer's disease and mild cognitive impairment. Acta Neuropathol (2010)] and brain imaging technologies [Petersen, R C., et al. Alzheimer's Disease Neuroimaging Initiative (ADNI): clinical characterization. Neurology 74:201-209 (2010)] are improving. However, the ability of these biomarkers to detect early stage disease has not been realized.

There are currently no universally accepted biomarkers in blood that correlate with disease progression in AD. Recent evaluation of a new kit assay designed to measure levels of various forms of Aβ protein in blood for possible use in early detection of Alzheimer's has been available for research use since the summer of 2007 (INNO-BIA plasma Aβ forms, Innogenetics, Gent Belgium). This test establishes an Aβ42/Aβ40 ratio that is lower in patients with a predisposition for developing mild cognitive impairment (MCI), which usually precedes AD. Unfortunately, peripheral Aβ measurements are subject to conflicting reports for a variety of reasons, [Cedazo-Minguez, A., and Winblad, B. Biomarkers for Alzheimer's disease and other forms of dementia: clinical needs, limitations and future aspects. Exp Gerontol 45, 5-14 (2010)]. A complex blood plasma molecular test for diagnosis of AD has recently been described, [Ray, S. et al. Classification and prediction of clinical Alzheimer's diagnosis based on plasma signaling proteins. Nat Med; 13: 1359-1362 (2007)] wherein 18 out of 120 signaling proteins were found with 90% accuracy to be predictive “markers” of AD. The statistical interpretations of these 18 signaling protein microarray proteins appears cumbersome and not readily converted to an easy to perform test.

### SUMMARY OF THE INVENTION

A laboratory method for screening, diagnosing, monitoring and/or staging early onset Alzheimer's disease which consists of mild cognitive impairment entails conducting a blood test after an oxidative exposure of serum to assay for the presence of an elevated level of redox-reactive autoantibodies.

A method of detecting or diagnosing early onset Alzheimer's disease in a subject, includes the steps of assaying an oxidized first blood sample from the subject to determine a baseline level of oxidized autoantibodies having selected specificities, treating a second longitudinal blood sample with an oxidizing agent and assaying the oxidized second sample to determine the level of autoantibodies having the selected specificities, and comparing the level of the autoantibodies in the first sample with the level of autoantibodies in the oxidized second sample, wherein an increase in the level of autoantibodies in the oxidized second sample as compared to the level of the oxidized first sample correlates with early onset Alzheimer's disease defined as mild cognitive impairment in said subject.

A blood serum biomarker for diagnosing, monitoring and/or staging early onset Alzheimer's disease defined as mild cognitively impairment comprising redox-reactive autoantibodies.

A kit for diagnosing, monitoring and/or staging early onset Alzheimer's disease defined as mildly cognitively impaired individuals includes a laboratory assay which can detect redox reactive autoantibodies before and after exposure to an oxidative agent.

### BRIEF DESCRIPTION OF THE DRAWINGS

FIG. 1. is a chart showing diagnostic treatment possibilities and evolution of irreversible dementia.

FIG. 2 shows a Classification And Regression Tree analysis (CART) of preliminary data results of applicant's R-RAA unmasked anti-PE ELISA in serum from MCI and normal donors (Values are OD units).

FIG. 3 shows hemin unmasked Redox Reactive Antibody Activity (R-RAA) in serum from normal age-matched controls and patients with mild cognitive impairment and Alzheimer's dementia.

### DETAILED DESCRIPTION OF THE INVENTION

It would be desirable to have a method, biomarker and kit for early diagnosis of AD using a simple blood test. Identification of a robust serum biomarker for identifying early asymptomatic AD will be essential for maximal efficacy of future therapeutics currently in clinical development aimed at both halting disease progression and/or modifying the rate of cognitive decline. The sensitivity of CSF biomarkers and brain imaging technologies to stage AD disease progression are improving, but at present there are no inexpensive laboratory tests that are clinically useful to identify at-risk individuals for developing AD. The determination of diagnostic accuracy for novel AD biomarkers requires studies of samples of bodily fluids obtained longitudinally from individuals until AD can be confirmed at autopsy. Applicant can demonstrate a serum biomarker from anamnestic mild cognitively impaired (MCI) subjects that can discriminate, at high levels of significance, serum samples derived from MCI patients versus those from AD patients and/or normal age-matched controls. A serum biomarker for the detection and staging of AD is an ideal minimally-invasive technique that can be used in routine annual screening.

The basis of applicant's AD biomarker invention is based on the discovery of a novel family of autoantibodies present in serum that have their antigen recognition sites “masked.” Oxidative unmasking of these redox-reactive autoantibodies (R-RAA) in serum samples in vitro in the presence of their recognition epitopes quantitatively measures concentration of unmasked antibodies in serum using an ELISA format. Applicant's R-RAA technology has the potential for revolutionizing the medical community's ability to identify at-risk MCI individuals by assessing their increase in R-RAA over their base line values and their increased ELISA reactivity compared to AD and/or normal age-matched individuals.

Using patented technology, the identification and validation of a novel serum biomarker assay for the purpose of developing an in vitro diagnostic (IVD) was attempted. The initial requirements used to guide the design, format and implementation of the IVD were based on the following: (a) the 1998 Consensus Report [Consensus report of the working group on molecular and biochemical markers of Alzheimer's disease The Ronald and Nancy Reagan Research Institute of Alzheimer's Association and the National Institute on Aging Working Group. Neurobiol Aging; 19: 109-116 (1998)]. “An ideal marker should have greater than 80% sensitivity and specificity for excluding other dementias and neurodegenerative processes; it should be reliable, reproducible, non-invasive, easy to perform, and inexpensive” (b) The reagent components used in the assay must be suitably stable; and (c) must incorporate applicant's proprietary technology in order to justify development, validation and implementation costs. Previous work by the present inventor demonstrated potential by using 16 AD serum samples and 17 age-matched serum samples from volunteer blood donors. Each serum was tested before and after unmasking of the R-RAA with hemin. R-RAA's specificities for PS, PC, CL and PE were evaluated by using an in-house ELISA. Comparisons between the AD and normal populations revealed highly significant differences in R-RAA antiphosphatidylethanolamine (aPE), McIntyre, J A, Wagenknecht, D R, and Ramsey, C J. Redox-reactive antiphospholipid antibody differences between serum from Alzheimer's patients and age-matched controls. Autoimmunity, 42:646-52, (2009). In-sample Fisher's linear discriminate analysis found a sensitivity of 88% and a specificity of 94%. In-sample Classification and Regression Tree analysis (CART) found a sensitivity of 84% and a specificity of 100%. This study was the first to indicate that blood tests for R-RAA may be used as a laboratory criterion for an Alzheimer's diagnosis. These preliminary data are encouraging because it met the 1998 Consensus report “that an ideal marker should have greater than 80% sensitivity”. Additionally, reduced serum ethanolamine plasmalogen in late stages of AD [Wood, P. L., Mankidy, R., Ritchie, S., Heath, D., Wood, J. A., Flax, J., and Goodenowe, D. B. Circulating plasmalogen levels and Alzheimer Disease Assessment Scale-Cognitive scores in Alzheimer patients. J Psychiatry Neurosci; 35:59-62 (2010)] indicate that serum biomarkers associated with AD are encouraging.

Taking a small sample of the patient's blood does not pose the problems or limitations for routine annual AD screening in the primary care physician's office encountered with sampling CSF by lumbar puncture. It is for this reason that identifying a reliable blood biomarker that strongly correlates with early neurodegenerative disease progression will revolutionize screening of individuals at risk for developing AD just as routine blood lipid panel testing has revolutionized treatment of the hyperlipidemias. Given the expected escalating incidence and prevalence of AD with an aging population as the 21^{st} century unfolds, it is easy to see why routine annual screening with a biomarker for AD would be highly desirable. Indeed one has only to see how Prostate Specific Antigen (PSA) screening has gained acceptance as a routine test, not because absolute PSA levels in a given patient are necessarily predictive, but a relative increase or trend over several years of single patient's PSA values can be a powerful early warning sign for development of prostate cancer warranting further more invasive biopsy evaluation. It is this added power of longitudinal sampling data from individuals that can substantially increase the predictive sensitivity of the biomarker. For these reasons the identification of a serum biomarker that robustly correlates with MCI in early AD has the potential to also be measurable in the late asymptomatic phase of the disease, where therapeutic intervention has the potential of reversing disease progression. The identification, validation and commercialization of such a serum biomarker for identifying asymptomatic at-risk individuals during a routine annual physical exam will be potentially ‘disruptive’ technology that must be quickly and expeditiously evaluated for implementation.

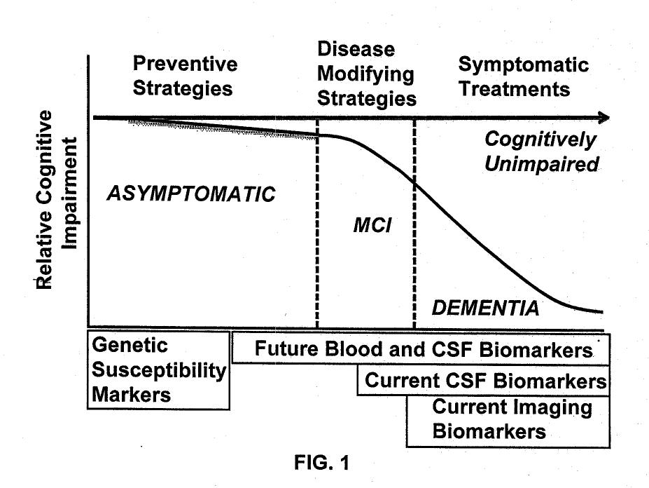

Identification of a robust biomarker for identifying early asymptomatic AD will be essential for future therapeutic interventions aimed at halting disease progression rather than simply modifying the rate of cognitive decline. With sporadic AD an individual is unlikely to become aware of mild cognitive impairment (MCI) until fulminate disease progression is established. As shown in FIG. 1, which shows diagnostic treatment possibilities and evolution of irreversible dementia, this is precisely the stage of AD where patients invariably compensate for cognitive problems and are often in denial of their symptoms to family and to their primary care physician. The ‘grayness’ scale cartoon at the top of the figure denotes the ‘probability’ of disease reversal by therapeutics. The ‘grayness scale’ cartoon in the middle of the figure represents a theoretical time window where therapeutics currently in clinical trials may afford the possibility for complete reversal of disease, or at least halting disease progression. (Figure modified from Cedazo-Minguez, A., and Winblad, B. Biomarkers for Alzheimer's disease and other forms of dementia: clinical needs, limitations and future aspects. Exp Gerontol 45, 5-14 (2010))

For this reason individuals are unlikely to voluntarily seek CSF biomarker assessment for AD diagnosis by requesting a lumbar puncture procedure. To really address the escalating incidence and prevalence of AD the discovery of a serological biomarker that could accurately detect early AD would solve this problem. If treatment options became available that arrest the disease when caught early, the general public, physicians and insurance companies alike would demand annual blood sample screening for AD to ward off the terrible consequences of living with the disease and its associated devastating financial and social costs. The 1998 Consensus Report of the Working Group on Molecular and Biochemical Markers of AD determined that an ideal marker should have a greater than 80% sensitivity and specificity for excluding other dementias and neurodegenerative processes, and it should be reliable, reproducible, non-invasive, easy to perform, and inexpensive [Consensus report of the working group on molecular and biochemical markers of Alzheimer's disease The Ronald and Nancy Reagan Research Institute of Alzheimer's Association and the National Institute on Aging Working Group. Neurobiol Aging 19:109-116 (1998)].

The present inventor has previously reported the discovery that blood and other bodily fluids from normal individuals contain a significant number of antibodies, that, when treated with an oxidizing agent, become capable of binding self antigens. See, for example, the following publications:
McIntyre, J A. “The appearance and disappearance of antiphospholipid antibodies subsequent to oxidation-reduction reactions.” Thromb. Res. 2004; 114:579-87.
McIntyre, J A, Wagenknecht, D R, & Faulk, W P. “Autoantibodies unmasked by redox reactions.” J. Autoimmun 2005; 24:311-17.
McIntyre, J A, Wagenknecht, D R, & Faulk, W P. “Redox-reactive autoantibodies: Detection and physiological relevance. Autoimm. Rev. 2006; 5:76-83. and U.S Patent Application Publication No. 2005/0101016 A1.
McIntyre, J A & Faulk W P. Redox-reactive autoantibodies: biochemistry, characterization, and specificities. Clin Rev Allergy Immunol 37, 49-54 (2009).
McIntyre, J A, Wagenknecht, D R, and Ramsey, C J. Redox-reactive antiphospholipid antibody differences between serum from Alzheimer's patients and age-matched controls. Autoimmunity, 42: 646-52 (2009).

The entire contents of each of these publications are incorporated herein by reference.

Such autoantibodies may be detected by treating the blood or other bodily fluid with an oxidizing agent and then using a screening assay to detect antibodies that bind a self antigen. It has been found that such autoantibodies are present in blood or other bodily fluids in a wide variety of isotypes and specificities. It has also been found that autoantibodies can be detected in a purified or fractionated immunoglobulin composition that has been treated with oxidizing agents. Since the autoantibodies are not detected above a minimal baseline in blood or other bodily fluids from normal individuals or in immunoglobulin compositions pooled from normal individuals in the absence of an oxidation step, antibodies or autoantibodies having this property are referred to herein as “masked” antibodies or “masked” autoantibodies, and the process of treating blood or other bodily fluids or immunoglobulin preparations with oxidizing conditions is referred to herein as “unmasking” the masked antibodies or autoantibodies. Antibodies having the property of becoming masked or unmasked, depending on oxidation-reduction conditions may also be referred to herein as “redox-reactive autoantibodies” (R-RAA).

The present inventor set out to develop a novel serological biomarker as a diagnostic tool with sufficient sensitivity and predictability to be clinically useful to identify asymptomatic individuals at-risk for developing early stage AD. R-RAA technology is based on the identification of disease-specific serum autoantibodies as a biomarker for disease progression [McIntyre, J. A., Wagenknecht, D. R., and Ramsey, C. J. Redox-reactive antiphospholipid antibody differences between serum from Alzheimer's patients and age-matched controls. Autoimmunity, 42:646-52 (2009)]. The present inventor has developed proprietary methods to ‘unmask’ these autoantibodies in serum samples in vitro and has further identified autoantibodies reactive to AD relevant epitopes.

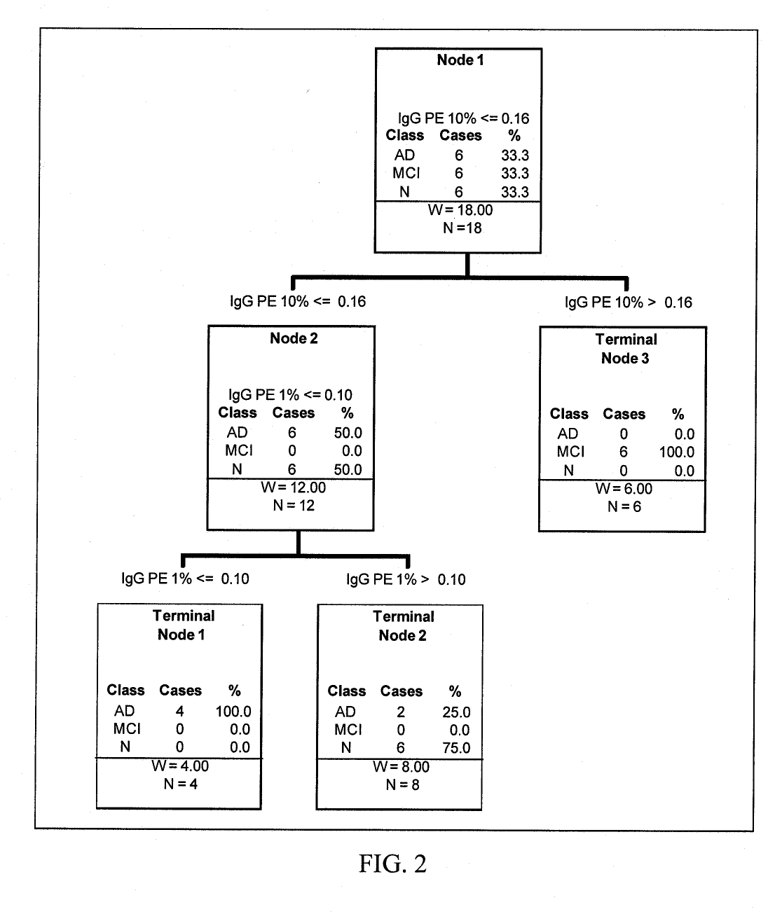

To further the AD studies, a pilot phase 1 study consisted of blinded sets of six samples from MCI, AD and cognitively normal donors (ND) were provided by the AD Neuroimaging Initiative (ADNI). These 18 coded frozen serum samples from ADNI were used for ‘blinded’ analysis of R-RAA aPE measurements. A totally unexpected observation emerged upon decoding the sample groups. In this study, as shown in the following Table and in FIG. 2, the MCI individuals had markedly elevated serum R-RAA aPE compared to NC (P=0.0003, FIG. 2).

t-Test: Two-Sample Assuming Unequal Variances

| | MCI | Normal |
|---|---|---|
| Mean | 0.219524 | 0.098046 |
| Variance | 0.00158 | 0.001535 |
| Observations | 6 | 6 |
| Hypothesized Mean | 0 | |
| Difference | | |
| df | 10 | |
| t Stat | 5.33164 | |
| P(T < = t) one-tail | 0.000166 | |
| t Critical one-tail | 1.812461 | |
| P(T < = t) two-tail | 0.000332 | |
| t Critical two-tail | 2.228139 | |

The mean R-RAA aPE values from the serum of confirmed AD cases were significantly reduced compared to the six control samples as well (P=0.011547). Of the six MCI patients, redox-reactive antibody technology correctly distinguished all 6 samples as serum originating from the MCI group. The increase in R-RAA aPE in MCI serum samples suggests that aPE autoantibodies may be generated in response to perturbations in lipid metabolism during MCI, but decline as the disease progresses to dementia. Although preliminary, the observed robust increase in aPE indicates that there may be sufficient detectable elevation of aPE during the asymptomatic phase of the disease to exploit R-RAA as a biomarker of early cognitive decline. 3R's findings can also be compared to the observed changes in hippocampal choline acetyltransferase (ChAT) activity [DeKosky, S. T., Ikonomovic, M. D., Styren, S. D., Beckett, L., Wisniewski, S., Bennett, D. A., Cochran, E. J., Kordower, J. H., and Mufson, E. J. Upregulation of choline acetyltransferase activity in hippocampus and frontal cortex of elderly subjects with mild cognitive impairment. Ann Neurol 51, 145-155 (2002)]. Only the end-stage AD group had ChAT levels reduced below normal controls On the other hand, hippocampal ChAT activity was significantly higher in MCI subjects than in either normal controls or AD. In summary, the significance of elevated R-RAA aPL in serum from MCI patients compared to NC formed the basis for continuing studies to evaluate the feasibility of the use of this approach as a novel serum biomarker for detection and staging of early AD.

To continue the MCI phase 2 studies, 90 additional blinded serum samples were provided by the ADNI that represented 30 cognitively normal age matched controls, 30 serum samples from putative MCI patients and 30 serum samples from patients diagnosed with Alzheimer's disease.

The oxidizing agent that was used in the development of the R-RAA aPE ELISA was hemin. Commercial sources of hemin, however, were found to give variable results in terms of time for unmasking and absolute ELISA OD values. Thus, Frontier Scientific Inc., Logan, Utah, has agreed to supply bulk quantities of hemin for manufacture of an in vitro diagnostic (IVD) to detect early onset Alzheimer's from normal individuals. The optimal dilution of the normal sera versus the final concentration of hemin is determined by checkerboard analyses. Historically, a 1/10 dilution of serum showed optimal unmasking of aPL after addition of 23 mM hemin and overnight incubation at 36° C. Serum dilution is required to counter the numerous components in the sera that can function as antioxidants. The detection of serum aPL before and after oxidation is assessed by using an in-house enzyme-linked immunosorbent assay (ELISA) that uses two specimen diluents, one containing 1% bovine serum albumin (BSA) in Tris-buffered saline (TBS) and the second diluent containing 10% adult bovine plasma (ABP) in TBS [McIntyre, J. A., Wagenknecht, D. R., and Waxman, D. W. Frequency and specificities of antiphospholipid antibodies (aPL) in volunteer blood donors. Immunobiology, 207:59-63 (2003)]. The BSA diluent allows detection of aPE that is independent of plasma-protein binding factors, whereas the ABP diluents allow detection of aPE that are dependent upon the binding of a plasma protein(s) to the phospholipids. The ELSA data are reported in raw OD units. The value from each sample is then matched to the group from which the sample was obtained by the provider of the samples. Statistical analyses of the data are then performed. Procedures for detection of R-RAA are provided as follows:

### Hemin Treatment of ADNI Sera Unmasks Redox-Reactive Anti-Phospholipid Autoantibodies

### Procedure #1:

A. Preparation of Serum Samples
(1) Aliquots of ADNI serum samples stored at −80° C. are thawed. Cleanascite™ (Biotech Support Group Inc. North Brunswick, N.J.) is suspended by gently rocking prior to use.
(2) 1:4 vol/vol of Cleanascite™ is added to each serum sample in a 2 ml micro tube, using a pipette tip with an orifice cut to ˜0.5 mm. The dilution factor of the serum is ˜1.125
(3) The samples are rocked at 37° C. by placing the tubes on their side on the platform of a Lab Line Titer-Plate Microplate Shaker in an incubator for 10 min and centrifuged for 1 min. at 16,000×g.
(4) The supernatants are carefully removed, and the samples are divided into two for ±treatment with hemin (see below).

(B) Preparation of Serum Dilution Buffer±Hemin.
(1) Hemin powder (Frontier Scientific, Logan Utah) is taken from storage at 4° C. (Cat. #H651-9 Batch FS108-50). A ˜112 mM stock solution of hemin (MW=651.96) is prepared by dissolving hemin powder in 1 M NaOH with warming and vigorous stirring.
(2) The solution is left to cool to room temperature for 2 hr and filtered through a 0.45 micron filter. The concentration of hemin is then accurately determined by using the mean mM extinction coefficient of hemin in NaOH of a sample appropriately diluted in 5 mM NaOH; 5.84 cm⁻¹ M⁻¹ at 385 nm (Li et al. Acta Biochimica et Biophysica Sinica, 2006; 38:63-69).
(3) The stock hemin solution is stable for at least 4 months at 4° C.
(4) Tris buffered saline buffer (pH 7.3±0.03) (TBS) is prepared as a 10× stock and diluted prior to use to 20 mM Tris base, 151 mM NaCl, 3 mM NaN₃.
(5) The stock hemin solution in NaOH is slowly added to the TBS buffer to a final concentration of 1.35 mM. Note volume of NaOH added. Adjust the pH of the hemin TBS buffer to 7.8±0.03 with 1M HCl.
(6) In a separate 100 ml beaker, add the same volume of TBS buffer, and add an equivalent volume of 1M NaOH that was used for the hemin solution. Adjust the pH to 7.8±0.03 with 1M HCl.
(7) The two aliquots of serum samples from each donor are diluted to a final of 1:15 vol/vol in either the hemin or TBS buffer ph 7.8. Note: The 1:15 dilution should take into account the 1.125 fold dilution of the serum samples following Cleanascite™ treatment. The samples are placed on an orbital shaker in an incubator at 37° C. for 3 hr then frozen at −80° C. for ELISA analysis.
(9) The samples are thawed, and diluted in either bovine serum albumin (BSA) or adult bovine plasma (ABP), diluted in TBS pH 7.3 at a final serum dilution of 1:50 for ELISA analyses. The BSA and ABP must be diluted in TBS such that the final concentrations of BSA and ABP in the ELISA are 1% and 10%, respectively.

### Procedure #2:

(1) Aliquots of ADNI serum samples stored at −80° C. are thawed and 100 ul added to 900 ul of both the hemin and NaOH control solution prepared as detailed above in B1-B6.
(2) The samples are incubated for 20 hr on a rocking platform at 36° C., then frozen at −80° C. for ELISA analysis.
(3) The samples are thawed and diluted in either bovine serum albumin (BSA) or adult bovine plasma (ABP), diluted in TBS, pH 7.3 at a final serum dilution of 1:100 for ELISA analyses. The BSA and ABP must be diluted in TBS to reach a final concentration 1% and 10%, respectively.
(4) The aPL ELISA method (McIntyre, et al. Immunobiology 2003; 207:59-63) is altered such that the IgG plates are stopped at 25 min and the IgA and IgM plates are stopped at 30 min.

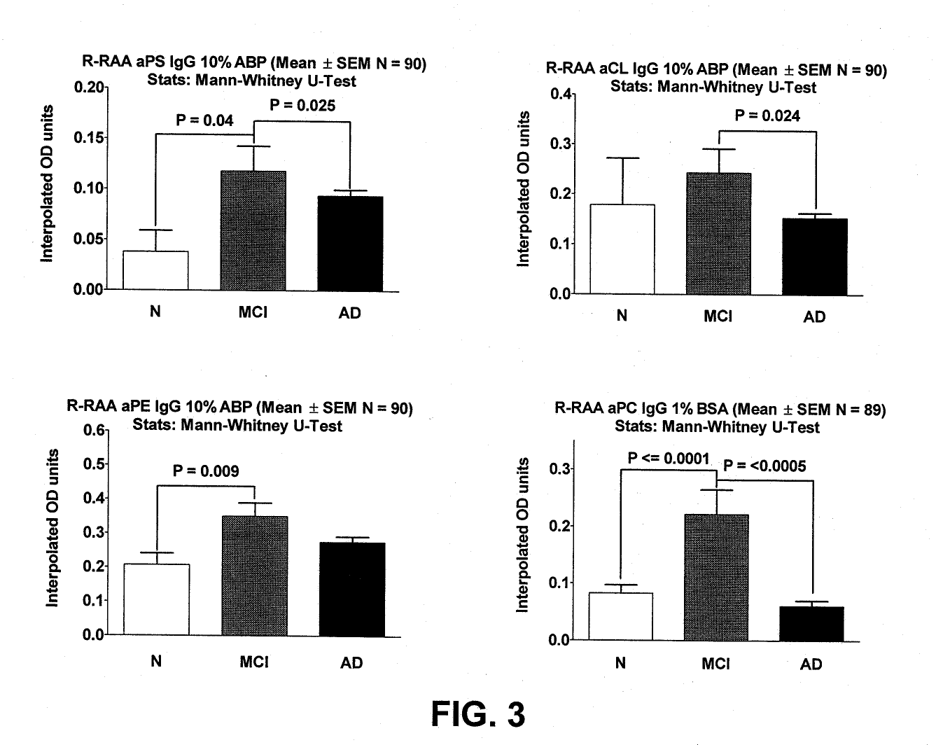

The new source of hemin from Frontier Scientific is 99% plus pure. This compared to the previous sources of hemin purchased from Sigma that were 80% and 90% pure. The differences in purity caused differences in the degree of unmasking as well as differences in reagent concentrations, temperatures, pH of the buffers and times of incubation. These changes are likely due to differences in the voltage potentials of the different source of hemin. Nonetheless, unmasking of aPL in serum continued to occur and the elevated unmasking of aPL in the MCI group of patent samples provided by the ADNI continued to be significantly higher in ELISA OD values than the normal and/or AD groups. An example of these aPL differences is shown in FIG. 3. In FIG. 3, ELISA OD values are interpolated from standard curves using qualified lots of anti-phospholipid antisera. Intrinsic antiphospholipid antibody activity (OD values of samples untreated with hemin) is subtracted from the hemin treated OD values to give the R-RAA activity in the serum samples.

Thus, aspects of the present invention provide a method of detecting, diagnosing and/or staging Alzheimer's disease (AD) in a subject. Further aspects of the invention provide a method of monitoring a subject over a period of time to detect the development or progress of unmasking aPL that are harbingers of MCI of the AD type.

These and other objectives are achieved by a method of detecting or diagnosing a neurodegenerative disease or condition in a subject by obtaining a sample of blood from the subject, treating the sample with an oxidizing agent and assaying the sample to determine the presence or absence of autoantibody in said sample, wherein an elevated presence of autoantibody correlates with a an at-risk profile for developing AD in said subject.

The objectives are further achieved by a method of detecting or diagnosing an MCI profile in a subject by assaying an oxidized first blood sample from the subject to determine a level of aPL autoantibodies of selected specificities (establishing a baseline), then treating a second sample drawn at a later date from the subject with an oxidizing agent and assaying the oxidized second sample to determine the levels of aPL autoantibodies with the selected specificities, and comparing the level of the autoantibodies in the oxidized first sample with the level of the autoantibodies in the oxidized second sample. For example, wherein there is a lack of increase in the levels of the oxidized second sample as compared to the levels in the first sample, this indicates that MCI in said subject does not appear to support impending AD. In contrast, if the second or subsequent samples show increased aPL reactivities, further testing is warranted as it may signal the beginning of MCI or early onset AD.

There is a critical unmet need to develop a reliable predictive biomarker for AD that will detect earlier disease onset and to more widely screen and identify at-risk individuals. Applicant's data indicate that R-RAA may have value as a biomarker that can be developed as a danger-early-warning sign of approaching AD and used as a sero-epidemiological tool. It might also be (more) useful as an approach to beginning to understand the pathophysiology of AD.

Further, it has been shown that blood contains at least the following masked aPL: anticardiolipin (aCL), antiphosphatidylcholine (aPC), antiphosphatidylethanolamine (aPE), and, antiphosphatidylserine (aPS).

According to one embodiment of the present invention, anamnestic MCI is diagnosed by obtaining a sample of blood from a subject, treating the sample with an oxidizing agent and assaying the oxidized sample for an elevated level of antiphospholipid autoantibodies compared to a previously determined baseline for this individual. The assay method that is used in this embodiment detects direct and indirect binding to a phospholipid (i.e. plasma protein dependent versus plasma protein independent binding), so that only aPL that are in an active or unmasked form are detected and so that masked autoantibodies are not detected.

Elevated levels of aPL may be determined by reference to a previously recorded baseline value. For example, the baseline value may be a level of autoantibodies previously obtained from an oxidized sample from the subject at a time when the subject did not have symptoms of a neurodegenerative disease or the baseline value may be an average or mean value of a level of aPL in a population of control individuals. For example, a baseline of antiphospholipid antibodies from 59 normal subjects is described in Sokol, D. K., et al. “Testing for antiphospholipid antibody (aPL) specificities in retrospective “normal” cerebral spinal fluid (CSF)”. Clin. Develop. Immunol.; 11:7-12 (2004).

According to another aspect of the present invention, an MCI condition is diagnosed in a subject by assaying an oxidized sample of blood from the subject for the presence and level of autoantibodies of selected specificities, and then treating a second sample of blood from the subject with an oxidizing agent and then assaying the oxidized sample for the presence and level of the same autoantibodies. In other words, the assays are carried out to determine if there is an increase in the level of specific autoantibodies after treatment with an oxidizing agent. As discussed previously, it has been found that in normal individuals, the levels of aPL autoantibodies detected in blood increase significantly after a sample is oxidized. As also discussed above, a significantly higher level of aPL can be found in the blood of MCI individuals that has been treated with an oxidizing agent such as hemin. These findings suggest that in a patient with early onset AD such as in MCI, circulating unmasked autoantibodies may have become increased as an initial compensatory reaction to the high levels of oxidative stress associated with AD [Ischiropoulos, H, and Beckman, J S. Oxidative stress and nitration in neurodegeneration: cause, effect, or association? J Clin Invest; 111:163-169 (2003)]. This also may provide an explanation as to why there is a decrease in the level of aPL in the blood of patients with Alzheimer's after treatment with an oxidizing agent. Thus, according to this embodiment of the present invention, a significant increase in the level of the aPL in the oxidized second sample as compared to the level of the aPL in the oxidized first sample correlates with an MCI condition in said subject.

In practicing this embodiment of the method of the present invention, for determining the antibody level of an untreated blood sample taken from a subject, the redox state of the sample should not be altered until it is intentionally oxidized. In other words, care should be taken to ensure that masked autoantibodies in the blood do not become unmasked by treatment steps after the sample is obtained and before the intentional oxidation step, leading to a false positive result, and that unmasked autoantibodies in the blood do not become masked by treatment steps after the sample is obtained, leading to a false negative result. In particular, the sample should not be exposed to oxidation or reduction (redox) conditions. Typically, normal sample handling procedures, including freezing and thawing, and typical binding assay conditions are sufficient to preserve the redox state of samples.

For determining the autoantibody level of a treated sample, the sample may be treated with an oxidizing agent by any of the methods described in the publications and patent applications referenced and/or incorporated by reference herein. As a non-limiting example, a sample may be treated with hemin as described in Table 1 shown above. Other oxidizing agents and other incubation temperatures may readily be determined by persons skilled in the art.

A biomarker comprising the redox-reactive autoantibodies can be used such that a change in the optical density values of as measured by an ELISA after exposing an individual's serum to an oxidizing agent (e.g., hemin) can be used for screening, diagnosing, monitoring and/or staging early onset Alzheimer's disease which consists of mild cognitive impairment (MCI). The MCI group has significantly higher OD values than do the normals. Therefore, if the MCI values for a given individual are higher than in previous longitudinal samples from this given individual, i.e., if there is a rise in the OD values of redox-reactive antiphospholipid autoantibodies in an individual over time, this may be a harbinger of approaching Alzheimer's disease. Thus the test of the present invention is like a PSA test or measuring cholesterol levels over time; if the values increase it is a danger early warning signal.

A kit for screening, diagnosing, monitoring and/or staging early onset Alzheimer's disease which consists of MCI can include the reagents needed to perform the test, i.e., an oxidizing agent and instructions how to use it. The kit may also include a positive control, a calibrator to figure extrapolation values, buffers that the patient serum sample is diluted into, a conjugate (color indicator) that is isotype specific, e.g. IgG, and a stopping reagent (usually a strong base like sodium hydroxide).

The invention claimed is:

1. A laboratory method for screening, diagnosing, monitoring and/or staging early onset Alzheimer's disease which consists of mild cognitive impairment comprises conducting a blood test after an oxidative exposure of serum to assay for the presence of an elevated level of redox-reactive autoantibodies.

2. The method of claim 1, wherein the antiphospholipid autoantibody is anticardiolipin, antiphosphatidylcholine, antiphosphatidylethanolamine or antiphosphatidylserine.

3. The method of claim 1 wherein the subject is selected as exhibiting physical or cognitive symptoms of an early onset Alzheimer's disease.

4. The method of claim 1, wherein the subject has a family history of Alzheimer's disease.

5. The method of claim 1, wherein the subject has a family history of Alzheimer's disease and wherein the subject is at or beyond an average age of onset of family members having said Alzheimer's disease.

6. The method of claim 1, wherein an elevated level of at least one antiphospholipid autoantibody is determined relative to a baseline value.

7. The method of claim 1, wherein an elevated level of redox-reactive autoantibodies is determined relative to a baseline value or wherein the baseline value is an average or mean value of a level found in a population of control individuals.

8. A method of monitoring the development or progress of early onset Alzheimer's disease over a period of time, wherein the method comprises carrying out the method of claim 1 at the beginning of a period of time and then carrying out repetitions of the method at subsequent times.

9. A method of detecting or diagnosing early onset Alzheimer's disease in a subject, the method comprising the steps of assaying an oxidized first blood sample from the subject to determine a baseline level of oxidized autoantibodies having a selected specificities, treating a second longitudinal blood sample with an oxidizing agent and assaying the oxidized second sample to determine the level of autoantibodies having the selected specificities, and comparing the level of the autoantibodies in the first sample with the level of autoantibodies in the oxidized second sample, wherein an increase in the level of autoantibodies in the oxidized second sample as compared to the level of the oxidized first sample correlates with early onset Alzheimer's disease defined as mild cognitive impairment in said subject.

10. The method of claim 9, wherein the autoantibodies having selected specificities are anticardiolipin, antiphosphatidylcholine, antiphosphatidylethanolamine or antiphosphatidylserine.

11. A blood serum biomarker for diagnosing, monitoring and/or staging early onset Alzheimer's disease defined as mild cognitively impairment comprising redox-reactive autoantibodies.

12. A kit for diagnosing, monitoring and/or staging early onset Alzheimer's disease defined as mildly cognitively impaired individuals which comprises a laboratory assay which can detect redox reactive autoantibodies before and after exposure to an oxidative agent.
